(19) United States
(12) Patent Application Publication
(10) Pub. No.: US 2014/0155371 A1
(43) Pub. Date: Jun. 5, 2014

(54) MULTICOMPONENT CRYSTALLINE SYSTEM OF EZETIMIBE AND PROLINE

(71) Applicants: Andreas Hafner, Gelterkinden (CH); Tobias Hintermann, Therwit (CH); Martin Szelagiewicz, Basel (CH); Bernd Siebenhaar, Kandern-Wollbach (DE); Fritz Blatter, Reinach (CH)

(72) Inventors: Andreas Hafner, Gelterkinden (CH); Tobias Hintermann, Therwit (CH); Martin Szelagiewicz, Basel (CH); Bernd Siebenhaar, Kandern-Wollbach (DE); Fritz Blatter, Reinach (CH)

(73) Assignee: BASF SE, Ludwigshafen (DE)

(21) Appl. No.: 14/234,158

(22) PCT Filed: Jul. 24, 2012
(86) PCT No.: PCT/IB12/53751

§ 371 Date: Jan. 22, 2014

Related U.S. Application Data
(60) Provisional application No. 61/511,578, filed on Jul. 26, 2011.

(30) Foreign Application Priority Data
Jul. 26, 2011 (EP) 11175340.6

(51) Int. Cl. C07D 205/08

(52) U.S. Cl. CPC C07D 205/08
USPC 514/210.02; 548/952

(57) ABSTRACT

Provided is a crystalline composition comprising a mixture of a compound of formula 1 (Ezetimibe) and proline or proline derivatives, or a hydrate/solvate thereof, as well as a process for obtaining the same. And a process for the purification of Ezetimibe is also disclosed.

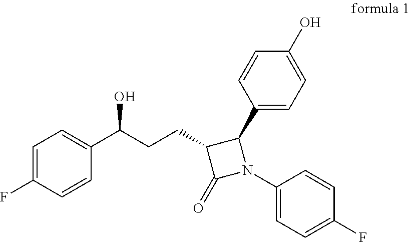 [SMILES: O=C1[C@H](CC[C@H](O)c2ccc(F)cc2)[C@@H](c2ccc(O)cc2)N1c1ccc(F)cc1]

The present invention relates to a multicomponent system comprising Ezetimibe and proline and to pharmaceutical preparations comprising said system, and specifically to a homogenous crystalline phase (co-crystal) comprising Ezetimibe and (S)-proline. The invention also relates to processes for preparing said multicomponent system and crystalline phase. The invention also relates to compositions comprising said multicomponent system or crystalline phase and a pharmaceutically acceptable carrier, and to methods of using said multicomponent system or crystalline phase to treat a disease condition wherein inhibition of the absorption of cholesterol from the small intestine is beneficial.

Ezetimibe, named 1-(4-Fluorophenyl)-3(R)-[3-(4-fluorophenyl)-3(S)-hydroxypropyl]-4(S)-(4-hydroxyphenyl)azetidin-2-one has the chemical structure of formula (1)

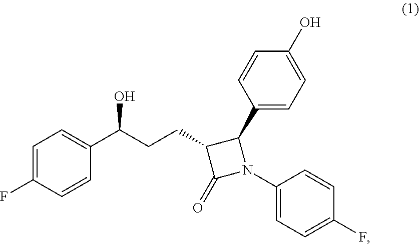 [SMILES: O=C1[C@H](CC[C@H](O)c2ccc(F)cc2)[C@@H](c2ccc(O)cc2)N1c1ccc(F)cc1]

It is known as a lipid lowering compound selectively inhibiting the intestinal absorption of cholesterol and related phytosterols. It is reported that the mechanism of action of Ezetimibe differs from that of other classes of cholesterol reducing compounds like HMG-CoA reductase inhibitors, fibric acid derivatives, bile acid sequestrants, and plant stanols (Kater et al. Diabetology & Metabolic Syndrome 2010, 2:34). Ezetimibe reportedly does not inhibit cholesterol biosynthesis or increase bile acid excretion. Instead, it appears that Ezetimibe is a specific cholesterol absorption inhibitor that acts at the brush border of the small intestine, blocking the absorption of dietary and biliary cholesterol and plant sterols, resulting in intracellular cholesterol depletion. This mechanism is complementary to that of HMG-CoA reductase inhibitors and adding Ezetimibe to statin therapy induces a 15% reduction in LDL levels compared with only 6% achieved by doubling the dose of statins (Sweeney et al. Expert Opinion on Drug Metabolism & Toxicology 2007, 3, 441).

Ezetimibe is sold under the brand name Zetia®, and in combination with Simvastatin under the brand name Vytorin®, both marketed by Merck/Schering Plough Pharmaceuticals. Zetia is available as a tablet for oral administration containing 10 mg of Ezetimibe and the following inactive ingredients: croscarmellose sodium NF, lactose monohydrate NF, magnesium stearate NF, microcrystalline cellulose NF, povidone USP, and sodium lauryl sulfate NF. Vytorin is available for oral use as tablets containing 10 mg of Ezetimibe, and 10 mg of Simvastatin (Vytorin 10/10), 20 mg of Simvastatin (Vytorin 10/20), 40 mg of Simvastatin (Vytorin 10/40), or 80 mg of Simvastatin (Vytorin 10/80). Each tablet contains the following inactive ingredients: butylated hydroxyanisole NF, citric acid monohydrate USP, croscarmellose sodium NF, hypromellose USP, lactose monohydrate NF, magnesium stearate NF, microcrystalline cellulose NF, and propyl gallate NF. Ezetimibe is useful in the treatment of hypercholesterolemia.

In WO 05/009955 are disclosed two crystalline forms, hereafter referred to as forms H1 and H2 of 1-(4-Fluorophenyl)-3(R)-[3-(4-fluorophenyl)-3(S)-hydroxypropyl]-4(S)-(4-hydroxyphenyl)azetidin-2-one, which are prepared either by dissolving Ezetimibe in an organic solvent such as acetone or ethylacetate under heating and then cooling the solution, adding a non solvent such as n-heptane, to precipitate crystalline form H1. Or by dissolving Ezetimibe in an organic solvent such as dioxane or acetonitrile under heating and then cooling the solution, optionally adding water, to precipitate crystalline form H2.

WO 06/060808 discloses two further crystalline forms of Ezetimibe referred to as anhydrous form A and hydrate form B as well as mixtures of form A and B and their preparation using several methods. Further documents disclosing certain crystalline forms of Ezetimibe are WO 05/062897, US-A-2006-0234996, IPCOM000131677D.

A single molecule, like Ezetimibe, may give rise to a variety of crystalline forms with different crystal structures and consequently different physical properties like melting point, X-ray diffraction pattern, thermal stability, hygroscopicity and solubility. The difference in the physical properties among crystalline forms is a result of the orientation and intermolecular interaction of adjacent molecules or complexes in the solid state. For pharmaceutically active ingredients, the solubility in aqueous solution, especially in the gastric juices of humans, is a physical property of fundamental importance, strongly influencing the compound's bioavailability.

The discovery of new crystal forms of a pharmaceutically useful compound offers an opportunity to improve the performance profile of a pharmaceutical product. It widens the reservoir of materials a formulation scientist has available for designing a new dosage form of a drug with improved characteristics.

Existing solid forms of Ezetimibe still leave room for improvement of physical as well as biological characteristics. There exists a need for other solid forms, especially crystalline forms, of 1-(4-Fluorophenyl)-3(R)-[3-(4-fluorophenyl)-3(S)-hydroxypropyl]-4(S)-(4-hydroxyphenyl)azetidin-2-one for sufficient diversity on crystalline materials to optimize manufacture, formulation, stability, and biological efficiency.

### SUMMARY OF THE INVENTION

The invention provides a novel crystalline solid form of Ezetimibe comprising proline and/or proline derivatives like 2-methylproline, 3-methylproline, 4-methylproline, 5-methylproline, N-methylproline, proline methylester, 4-hydroxyproline, 3,4-dehydroproline, and, consequently, novel pharmaceutical formulations containing this form. The invention further provides processes for manufacture thereof.

Crystalline forms often show desired different physical and/or biological characteristics which may assist in the manufacture or formulation of the active compound, to the purity levels and uniformity required for regulatory approval. The present solid form, especially crystalline form, possesses improved pharmacological characteristics, for example, improved bioavailability, thus offering enhanced possibilities to modulate and design improved drug products. Moreover, the tendency of the composition of the present invention to form interconvertible hydrates upon changing the relative humidity is much lower compared with solid forms of Ezetimibe known in the art.

### DETAILED DESCRIPTION OF THE INVENTION

The present invention is directed to a crystalline composition comprising a mixture of a compound of formula 1 (INN: Ezetimibe)

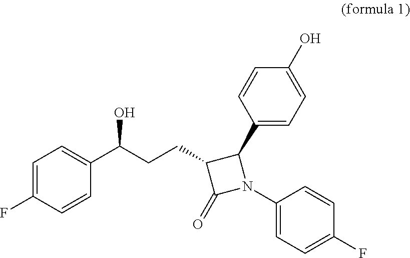 [SMILES: O=C1[C@H](CC[C@H](O)c2ccc(F)cc2)[C@@H](c2ccc(O)cc2)N1c1ccc(F)cc1]

and a second component selected from proline and proline derivatives, or a solvate (or hydrate) of said crystalline composition.

Solvates are generally crystalline compositions of the invention which contain, besides Ezetimibe and the second component (proline/proline derivative), water or a water miscible solvent as identified further below, especially preferred are solvates containing water, i.e. hydrates.

Useful proline derivatives in the crystalline composition of the invention are, for example, 2-methylproline, 3-methylproline, 4-methylproline, 5-methylproline, N-methylproline, proline methylester, 4-hydroxyproline, 3,4-dehydroproline. Hence, typical second components are

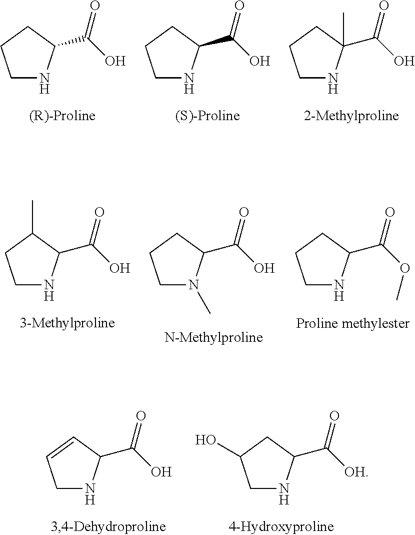

It has been the finding of the present invention that Ezetimibe is able to form a single crystalline phase (i.e. forming a co-crystal) together with proline or proline derivatives.

Preferably, the molar ratio of the compound of formula 1 and proline/proline derivative is the range of from 1:0.5 to 1:2.5, especially 1:0.9 to 1:2.1. Preferably, the single crystalline phase (i.e. co-crystal) contains the compound of formula 1 and proline/proline derivative (especially proline) in the molar ratio ranging from 0.9 to 2.1 molar parts of proline/proline derivative on 1 molar part of Ezetimibe. Of special importance is the co-crystal of approximate 1:1 stoichiometry, i.e. containing the compound of formula 1 and proline/proline derivative (especially proline) in the molar ratio ranging from 0.9 to about 1.1 molar parts of proline/proline derivative on 1 molar part of Ezetimibe.

Preferably, the second component (i.e. proline/proline derivative) is proline and is selected from (S)-proline (L-proline) or (R)-proline (D-proline). The crystalline composition of the invention thus preferably essentially consists of Ezetimibe (i.e. the compound of formula 1) and proline, besides minor amounts of water

In a further preferred embodiment, the crystalline composition is characterized in that proline is (S)-proline (L-proline) and it has an XRPD pattern with at least one characteristic peak (expressed in 2θ±0.2°2θ (CuKα radiation)) selected from 12.9, 16.3, 17.0, 19.0, 20.0, 22.4, and 24.5°; and typically showing all of these peaks. Preferably, it has an XRPD pattern with at least one characteristic peak (expressed in 2θ±0.2°2θ (CuKα radiation)) 9.5, 12.9, 16.3, 16.7, 17.0, 19.0, 20.0, 22.4, 24.5, 25.3°; typically showing all of these peaks. An XRPD pattern is shown in FIG. 1.

In a further preferred embodiment, the crystalline composition comprising Ezetimibe and (S)-proline has Raman bands at 2950, 1738, 1612, 1391 and 846 cm⁻¹, all within an accuracy of 2 cm⁻¹. Preferably, the crystalline composition has Raman bands at 3078, 3062, 2950, 2856, 1738, 1612, 1512, 1391, 1168, 1156, 846, and 637 cm⁻¹, all within an accuracy of 2 cm⁻¹. A FT Raman spectrum of the crystalline composition in the range from 1800 to 200 cm⁻¹ is shown in FIG. 2 and in the range from 3200 to 2700 cm⁻¹ in FIG. 3.

Preferably, the crystalline composition has a molar ratio of the compound of formula 1 and (S)-proline in the range of from 1:0.9 to 1:1.1, hereinafter designated as crystalline composition A.

(S)-Proline and Ezetimibe are present in the same solid phase as a homogeneous solid phase, i.e. forming a co-crystal. The invention thus further pertains to a novel crystalline form of Ezetimibe, which crystalline form is characterized by containing (S)-proline within its crystalline structure, e.g. in amounts as indicated above. A preferred novel crystalline form generally exhibits a characteristic X-ray powder diffraction pattern.

Another object of the invention is a process for obtaining the crystalline composition comprising the steps of:

a) providing a compound of formula 1 (INN: Ezetimibe)

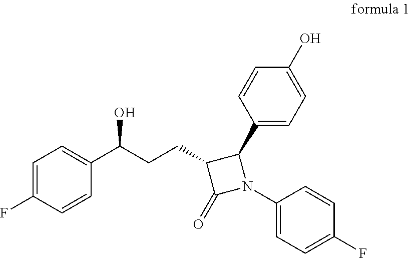 [SMILES: O=C1[C@H](CC[C@H](O)c2ccc(F)cc2)[C@@H](c2ccc(O)cc2)N1c1ccc(F)cc1]

in a suitable solvent or a mixture of solvents;

b) adding proline or proline derivative to the mixture of step (a);

c) optionally concentrating the composition of step (b) and/or adding an antisolvent;

d) crystallizing;

e) optionally equilibrating the obtained suspension of step (d); and

f) isolating the obtained precipitate.

Preferably, the molar ratio of the compound of formula 1 in step (a) and the proline of step (b) is in the range from 1:0.5 to 1:3.

Preferably, in step (b) proline is added, especially (S)-proline (L-proline) is added.

Step (b) usually comprises providing (S)-proline in solid form, or as a solution of (S)-proline in water, or water containing a water miscible solvent, or an organic solvent in the absence of water (water-free solvent), as defined for step (a) below.

The solvent used in step (a) is water or a water miscible organic solvent such as an alcohol (e.g. methanol, ethanol, propanol, butanol), or an at least partially water miscible solvent like an ester (such as ethyl acetate, methyl acetate), ethers such as methyltert.butylether, or an aliphatic ketone (e.g. acetone, methyl ethyl ketone), or mixture of such solvents, or such a solvent with water. Solutions or suspension according to steps a) and/or b) preferably are concentrated solutions. Preferably, the solvent is selected from the group consisting of C1-C4 alcohols, a C3-C6 ketone, an ether or an acetic ester C1-C4 alkylester, acetonitril, a hydrocarbon or mixtures thereof.

In a further preferred embodiment in step (d) and/or (e), seed crystals are added.

The concentration of Ezetimibe may range from 0.1 to about 300 mg/ml of solvents (including water), preferably from 5 to 200 mg/ml.

The process is preferably carried out in the temperature range 15-70° C., especially 15-50° C., for example at ambient temperature. In a preferred process, step (c) is carried out at a temperature from the range 20-70° C., especially 20-60° C., or the mixture is heated to a temperature from said range, e.g. about 50° C. As an antisolvent, an organic solvent of low polarity may be added (e.g. selected from hydrocarbons, especially medium-chain alkanes such as heptane). The suspension thus tempered is then preferably cooled before step d). In a preferred process, the step is accompanied by seeding with crystals of the desired form (e.g. 1-10% b.w. of the total amount of Ezetimibe) at a temperature of about 20-50° C.

Ambient temperature means in the context of the invention a temperature range at room temperature, comprising 20 to 30° C. and preferably about 20 to 25° C.

The crystalline composition is isolated by filtering off the crystals and drying, e.g. in vacuum, an inert gas flow or both at ambient temperature, or elevated temperatures up to 60° C.

The crystalline composition is thermodynamically stable and can be dried at elevated temperatures, e.g. below 80° C., and is obtained as a fine powder with typical particle size distributions with the median size between 1 and 50 μm, preferably between 1 to 10 μm. This particle size range ensures a fast dissolution profile, while retaining the favorable handling properties in the formulation process.

Dynamic (water) vapor sorption (DVS) is a method well known in the art to monitor the adsorption of water on a solid material. Therefore, DVS is a suitable method to determine the hygroscopic nature of a pharmaceutical active ingredient.

The crystalline form A described in WO 06/060808 (i.e. the free base) and the present composition (as obtained in example 3 further below) are subjected to a DVS experiment; results are shown in FIG. 4 and in the below Table 1. The crystalline composition is less prone to water uptake under humidity, and is easy to formulate compared to the crystalline anhydrous (“free base”) form of Ezetimibe (see Table 1).

TABLE 1

| | | Crystalline Ezetimibe |
|---|---|---|
| | crystalline | free base (Form A of |
| | composition A | WO 06/060808) |
| Water vapor sorption: | 0.3% | 4.0% |
| water content after 10 h at 50% | | |
| r.h. | | |
| Water vapor sorption: | 0.8% | 4.3% |
| water content after 4 h at 60% | | |
| r.h. | | |

The composition of the present invention, and especially crystalline composition A, may contain minor amounts of water.

The prior art suggests that Ezetimibe shows a remarkable tendency to form polymorphs and solvates. In particular, the observed hydrate formation is undesirable because both the monohydrate and the anhydrate are interconvertible upon changing the relative humidity. The dashed line of FIG. 4 shows, that an exposure of the anhydrous form to an increasing ambient humidity results in hydrate formation, starting at a relative humidity of about 50%. When the hydrate form thus obtained is subjected to decreasing humidity, it begins to lose water below about 25% of relative humidity. Present data show that neither the anhydrous form nor the hydrate form are thermodynamically stable under common relative humidity conditions, which typically may range from about 15% relative humidity to 90% relative humidity. In addition, FIG. 4 shows that the conversion of Ezetimibe free base is not kinetically hindered; i.e., neither the anhydrate, nor the monohydrate are kinetically stable.

Such a conversion does not occur in the crystalline composition of the present invention.

The crystalline composition of the present invention may be used in pharmaceutical compositions in the same way as other forms of Ezetimibe previously known. Additionally, the present crystalline composition may be employed as an intermediate or starting material to produce the pure active ingredient (especially the active ingredient combined with the present second component, but reduced concentrations of other undesired components), e.g. in form of crystalline composition A. The present invention thus further provides a method for the purification of Ezetimibe, which method is characterized by the step of precipitating and/or isolating the co-crystal of Ezetimibe and proline or proline derivative, e.g. as foreseen by steps d) and/or f) of the process for obtaining the crystalline composition described above. This method of the invention preferably employs (S)-proline as the co-crystal former with Ezetimibe. The co-crystal is most preferably of the composition described above, and in the present examples, as composition A.

Thus, in a general sense, the present invention pertains to a composition comprising a compound of the formula 1 (i.e. Ezetimibe) and proline or a proline derivative such as 2-methylproline, 3-methylproline, 4-methylproline, 5-methylproline, N-methylproline, proline methylester, 4-hydroxyproline, 3,4-dehydroproline.

While Ezetimibe of formula 1 (1-(4-Fluorophenyl)-3(R)-[3-(4-fluorophenyl)-3(S)-hydroxypropyl]-4(S)-(4-hydroxyphenyl)azetidin-2-one, hereinafter referred to as the R,S,S-form) shows a strong tendency to crystallization together with (S)-proline or derivatives thereof, (R)-proline and derivatives thereof do not. On the other hand, undesired stereoisomers of Ezetimibe, such as the counterenantiomer 1-(4-Fluorophenyl)-3(S)[3-(4-fluorophenyl)-3(R)-hydroxypropyl]-4(R)-(4-hydroxyphenyl)azetidin-2-one (S,R,R-form) or the typical main contaminant of Ezetimibe (i.e. the R,R,S-form) do not show any comparable tendency towards crystallization with (S)-proline or its derivatives.

Thus, the composition comprising (S)-proline or its derivatives together with a contaminated Ezetimibe, typically in form of a solution, may conveniently be separated into a solid comprising the desired Ezetimibe (R,S,S-form) and a supernatant containing the unwanted diastereomers.

The purification process conveniently follows the same steps (a) to (f) as described above for the crystallization of the present crystalline composition A. For use as a medicament, the thus obtained composition A may be employed; if desired, however, proline or its derivative may conveniently be separated again using conventional separation techniques known in the art.

The present invention is also directed to a pharmaceutical composition comprising the crystalline composition and optionally one or more pharmaceutically acceptable excipients.

The amount of solid (especially crystalline) forms of 1-(4-Fluorophenyl)-3(R)-[3-(4-fluorophenyl)-3(S)-hydroxypropyl]-4(S)-(4-hydroxyphenyl)azetidin-2-one and hydrates thereof substantially depends on type of formulation and desired dosages during administration time periods. The amount in an oral formulation may be from 0.1 to 200 mg, preferably from 0.5 to 100 mg, and more preferably from 1 to 50 mg.

A solid pharmaceutical composition comprising the crystalline composition of the invention along with further excipients is generally characterized by at least one characteristic peak in an x-ray powder diffractogram (expressed in 2θ±0.2°2θ (CuKα radiation)) selected from 12.9, 16.3, 17.0, 19.0, 20.0, 22.4, and 24.5°.

Oral formulations may be solid formulations such as capsules, tablets, pills and troches, or liquid formulations such as aqueous suspensions, elixirs and syrups. Solid and liquid formulations encompass also incorporation of the present crystalline composition into liquid or solid food.

The crystalline composition according to the invention may be directly used as powders (micronized particles), granules, suspensions or solutions, or they may be combined together with other pharmaceutically acceptable ingredients in admixing the components and optionally finely divide them, and then filling capsules, composed for example from hard or soft gelatin, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, disintegrants, lubricants, surfactants, sweetening and other flavouring agents, coating materials, preservatives, dyes, thickeners, adjuvants, antimicrobial agents and carriers for the various formulation types.

Examples for binders are gum tragacanth, acacia, starch, gelatin, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyimide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan, carragenan or hyaluronic aid.

Examples for excipients are phosphates such as dicalcium phosphate.

Examples for disintegrants are croscarmellose sodium, crospovidone, low-substituted hydroxypropyl cellulose, sodium starch glycolate or alginic acid.

Examples for lubricants are natural or synthetic oils, fats, waxes, or fatty acid salts like magnesium stearate.

Surfactants may be anionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Examples for sweetening agents are sucrose, fructose, lactose or aspartam.

Examples for flavouring agents are peppermint, oil of wintergreen or fruit flavours like cherry or orange flavour.

Examples for coating materials are gelatin, wax, shellac, sugar or biological degradable polymers.

Examples for preservatives are methyl or propylparabens, sorbic acid, chlorobutanol, phenol and thimerosal.

Examples for adjuvants are fragrances.

Examples for thickeners are synthetic polymers, fatty acids and fatty acid salts and esters and fatty alcohols.

Examples for liquid carriers are water, alcohols such as ethanol, glycerol, propylene glycol, liquid polyethylene glycols, triacetin and oils. Examples for solid carriers are talc, clay, microcrystalline cellulose, silica, alumina and the like.

The formulation according to the invention may also contain isotonic agents, such as sugars, buffers or sodium chloride.

The crystalline composition according to the invention may also be formulated as effervescent tablet or powder, which disintegrate in an aqueous environment to provide a drinking solution.

A syrup or elixir may contain the crystalline composition of the invention, sucrose or fructose as sweetening agent a preservative like methylparaben, a dye and a flavouring agent.

The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of Ezetimibe whereupon the properties that distinguish the solid forms of Ezetimibe are lost. However, the use of the novel forms to prepare such solutions is considered to be within the contemplation of the invention.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Slow release formulations may also be prepared from the crystal form according to the invention in order to achieve a controlled release of the active agent in contact with the body fluids in the gastro intestinal tract, and to provide a substantial constant and effective level of the active agent in the gastric juice. The crystalline composition may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

The crystalline composition of the invention is also useful for administering a combination of therapeutic effective agents to an animal. Such a combination therapy can be carried out in using at least one further therapeutic agent which can be additionally dispersed or dissolved in a formulation.

The crystalline composition of this invention and its formulations respectively can be also administered in combination with other therapeutic agents that are effective to treat a given condition to provide a combination therapy.

The crystalline composition and the pharmaceutical composition according to the invention are highly suitable for effective treatment of disorders in connection with need of inhibiting the intestinal uptake of cholesterol and related phytosterols. Crystalline compositions of this invention and pharmaceutical compositions are especially useful in the treatment of hypercholesterolemia.

The crystalline composition and the pharmaceutical composition according to the invention are particularly suitable for administration in combination with therapeutic agents that inhibit the HMG-CoA reductase, subsequently suppressing the biosynthesis of cholesterol.

An object of the invention is also a therapeutic method for producing an intestinal cholesterol and related phytosterole uptake inhibiting effect in a mammal comprising administering to a mammal in need of such therapy, an effective amount of the present crystalline composition

The crystalline composition of the invention may be used as single component or as mixtures with other solid forms, which may be crystalline or amorphous. As to the novel crystalline composition of Ezetimibe it is preferred that these contain at least 25% by weight, especially at least 50% by weight, based on the total amount of Ezetimibe. Preferably, such an amount of the crystalline composition comprising Ezetimibe is at least 75% by weight, especially at least 90% by weight. Highly preferred is an amount of at least 95% by weight.

Another object of the invention is a method of delivering the crystalline composition to a host, which method comprises administering to a host an effective amount of the crystalline composition according to the invention.

A further object of the invention is the use of the for the manufacture of a medicament useful in the treatment of disorders in connection with need of inhibiting the intestinal uptake of cholesterol and related phytosterols, and subsequently suppressing the intestinal absorption of cholesterol, and especially useful in the treatment of hypercholesterolemia in a mammal, such as a human; and the solid forms according to the invention for use in medical therapy.

Preferably, the present invention is directed to the crystalline composition for use in the treatment of disorders in connection with need of inhibiting the intestinal uptake of cholesterol and related phytosterols, and subsequently suppressing the intestinal absorption of cholesterol, and especially useful in the treatment of hypercholesterolemia in a mammal, such as a human; and the solid forms according to the invention for use in medical therapy.

The following examples illustrate the invention.

Wherever noted, room temperature depicts a temperature from the range 20-25° C.; percentages are given by weight, if not indicated otherwise.

Abbreviations:

DMSO dimethyl sulfoxide

DVS Dynamic (water) vapor sorption

HPLC high pressure liquid chromatography

NMR nuclear magnetic resonance

FTIR Fourier-transformation infrared spectrometry

r.h. relative humidity (air, if not indicated otherwise)

TG thermogravimetry

v/v volume by volume

XRPD Powder X-ray diffraction

Instrumental

XRPD:

The measurements are carried out with a Stoe Stadi P and Mythen1K Detector and Cu-Kα1 radiation. Standard measurement conditions: transmission; 40 kV and 40 mA tube power; curved Ge monochromator; 0.02°2θ step size, 12 s step time, 1.5-50.5°2θ scanning range; detector mode: step scan; 1°2θ detector step; standard sample preparation: 10 to 20 mg sample is placed between two acetate foils; sample holder: Stoe transmission sample holder; the sample is rotated during the measurement.

Generally, the 20 values are accurate within an error of ±0.1-0.2°. The relative peak intensities can vary considerably for different samples of the same crystalline form because of different preferred orientations of the crystals.

Thermogravimetry coupled to infrared spectroscopy (TG-FTIR):

Thermogravimetry coupled with FT-infrared spectroscopy is a well known method that allows to monitor the mass loss of a given sample upon heating while identifying the volatile substances by infrared spectroscopy. Therefore, TG-FTIR is a suitable method to identify solvates or hydrates.

TG-FTIR is performed on a Netzsch Thermo-Microbalance TG 209, which is coupled to a Bruker FT-IR Spectrometer Vector 22 or IFS 28. The measurements are carried out using aluminum crucibles with a micro pinhole under a nitrogen atmosphere and at a heating rate of 10° C./min over the range 25-250° C.

¹H-NMR:

The ¹H-NMR spectra are recorded on a Bruker DPX 300 spectrometer.

Solvent: Deuterated methanol.

Raman Spectroscopy:

FT-Raman spectroscopy is performed using a Bruker RFS100 (Nd: YAG 1064 nm excitation, 300 mW laser power, Ge detector, 64 scans, range 25-3500 cm⁻¹, 2 cm⁻¹ resolution).

DVS:

Dynamic (water) vapour sorption (DVS) is performed with a Surface Measurement Systems Ltd. DVS-1 water sorption analyzer or with SPS11-100n moisture sorption instrument from Projekt Meβtechnik, Ulm, Germany.

Program: The relative humidity is kept at starting value of 0% for 5 hours, then continuously scanned from 0% to 60%, kept constant at 60% for 4 hours, and then scanned to 0% relative humidity, and kept constant for 4 hours. The scanning change rate of relative humidity is 5% per hour.

Experimental

Solvents: For all experiments, Fluka or Sigma Aldrich grade solvents are used. Selected solvents are dried using 3 or 4 A molecular sieves.

### EXAMPLES

### Example 1

### Preparation of Seed Crystals

A mixture of 102 mg Ezetimibe (mixture of hydrate and anhydrate forms) and 29 mg L-proline (Sigma #81709) is ground in an agate mortar at room temperature in the presence of about 50 microliters of methanol (≧99.9%) and air dried (solvent drop grinding). This solvent drop grinding procedure is carried out a total of three times. The solid material was characterized by XRPD.

### Example 2

### Preparation of Seed Crystals

205 mg of Ezetimibe (mixture of hydrate and anhydrate forms) is dissolved in 2.0 mL ethanol (≧99.8%) at room temperature. 58 mg of L-proline (Sigma #81709) are added. The suspension is sonicated for 1 minute and stirred at room temperature for 2 hours. While stirring at room temperature the suspension is seeded twice with approx. 5 mg of the 1:1 co-crystal of Ezetimibe with L-proline. The suspension is filtered and air dried for 2 minutes at room temperature. H-NMR shows a molar ratio Ezetimibe to L-proline of 1:1. The solid material was characterized by XRPD and a XRPD pattern of the Ezetimibe-L-Proline co-crystal as shown in FIG. 1 was obtained. Said form is referred to as crystalline composition A.

### Example 3

### Preparation at the 0.4g Scale

409 mg of Ezetimibe (mixture of hydrate and anhydrate forms) and 116 mg of L-proline (Sigma #81709) are dissolved in 4.0 mL ethanol (≧99.8%) at 70° C., stirred at 70° C. for 30 minutes and cooled to 50° C. while stirring with a magnetic stirrer. The solution is seeded with approx. 5 mg of the 1:1 co-crystal of Ezetimibe with L-proline (example 2). 4.0 mL heptane are added to the turbid solution. The suspension formed is cooled to 40° C. and seeded again with approx. 5 mg of the 1:1 co-crystal of Ezetimibe with L-proline. The suspension is further cooled to 27° C., sonicated for 1 minute and diluted with 8.0 mL of ethanol (≧99.8%):heptane 1:1 v/v. The suspension is seeded again with approx. 2 mg of the 1:1 co-crystal of Ezetimibe with L-proline and stirred for 14 hours at room temperature. The suspension is then filtered, air dried for 5 minutes, dried at room temperature/30 mbar for 30 minutes and at 50° C./30 mbar for 35 minutes. Yield: 344 mg (64%). ¹H-NMR spectroscopy indicates a molar ratio of Ezetimibe to L-proline of 1:1. Furthermore, the solid material is characterized by XRPD, FT-Raman, TG-FTIR and DVS. A XRPD pattern of the Ezetimibe-L-Proline co-crystal as shown in FIG. 1 (table 2) is obtained, and the Raman spectrum obtained from this material is shown in FIG. 2 and in FIG. 3 (table 3). Thermogravimetry coupled with FT infrared spectroscopy does not reveal any significant mass loss of the sample below 200° C. This result shows that the obtained co-crystal is an anhydrous, nonsolvated form. A comparative analysis of the water adsorption (by DVS) with crystalline anhydrous Ezetimibe shows that the water uptake at 60% relative humidity is about a factor of five lower for the Ezetimibe-L-proline co-crystals as compared with the crystalline anhydrous form of Ezetimibe. The material obtained in this example shows a water uptake of about 0.8% (bold line) whereas the crystalline anhydrous form of Ezetimibe (dashed line) shows a water uptake of about 4.3%. The DVS analysis is shown in FIG. 4.

Here and in the following the abbreviations in brackets mean: (vs)=very strong intensity; (s)=strong intensity; (m)=medium intensity; (w)=weak intensity; (vw)=very weak intensity.

TABLE 2
Powder X-ray diffraction peaks for the co-crystal.

| Pos. [°2θ.] | d-spacing [Å] | Qualitative Intensity |
|---|---|---|
| 9.5 | 9.3 | w |
| 12.9 | 6.9 | m |
| 14.2 | 6.3 | vw |
| 15.4 | 5.76 | w |
| 16.3 | 5.44 | vs |
| 16.7 | 5.32 | m |
| 17.0 | 5.20 | s |
| 19.0 | 4.66 | s |
| 19.3 | 4.60 | w |
| 20.0 | 4.43 | s |
| 20.1 | 4.40 | m |
| 21.4 | 4.14 | w |
| 21.7 | 4.09 | w |
| 22.4 | 3.96 | s |
| 22.6 | 3.92 | w |
| 22.9 | 3.89 | w |
| 24.1 | 3.70 | w |
| 24.5 | 3.63 | s |
| 24.7 | 3.60 | w |
| 24.9 | 3.57 | w |
| 25.3 | 3.52 | m |
| 25.9 | 3.44 | vw |
| 26.6 | 3.35 | w |
| 27.1 | 3.29 | vw |
| 27.3 | 3.27 | w |
| 29.8 | 3.00 | w |
| 30.2 | 2.96 | w |
| 30.5 | 2.93 | vw |
| 30.9 | 2.89 | w |
| 33.6 | 2.67 | w |

TABLE 3
Raman peaklist for the co-crystal

| Wavenumbers [cm−1] | Relative intensity |
|---|---|
| 3078 | M |
| 3062 | M |
| 3008 | W |
| 2950 | M |
| 2856 | W |
| 1738 | W |
| 1612 | Vs |
| 1512 | M |
| 1442 | W |
| 1391 | S |
| 1327 | W |
| 1292 | W |
| 1224 | W |
| 1168 | W |
| 1156 | M |
| 1098 | Vw |
| 1058 | Vw |
| 960 | Vw |
| 914 | Vw |
| 846 | M |
| 828 | Vw |
| 801 | Vw |
| 637 | M |
| 399 | Vw |
| 384 | Vw |
| 335 | Vw |
| 243 | Vw |

### Example 4

204 mg of Ezetimibe (mixture of hydrate and anhydrate forms) and 116 mg of L-proline (Sigma #81709) are suspended in 2.0 mL ethanol (≧99.8%), heated to 70° C., stirred for 30 minutes at 70° C. and cooled to room temperature while stirring with a magnetic stirrer and stirred overnight at room temperature. 2.0 mL ethanol (≧99.8%) are added to the suspension. The suspension is sonicated for 1 minute, stirred again for 3 hours at room temperature, filtered, air dried for 3 minutes and dried at room temperature/30 mbar for 30 minutes. Yield: 165 mg (52%). ¹H-NMR shows a molar ratio of Ezetimibe to L-proline of about 1:2. The solid material is characterized by XRPD. The XRPD pattern of the 1:2 co-crystal shows the same peaks as the 1:1 co-crystal with a small excess of L-proline.

### Example 5

102 mg of Ezetimibe (mixture of hydrate and anhydrate forms) and 29 mg of D-proline (Sigma-Aldrich #85891-9) are dissolved in 1.0 mL ethanol (≧99.8%) at 70° C., stirred at 70° C. for approximately 1 hour and cooled to room temperature while stirring with a magnetic stirrer. The solution is sonicated for 1 minute and 1.0 mL heptane is added. The solution is sonicated again for 1 minute and stirred for approximately 2 hours. The suspension formed is sonicated for 1 minute, filtered and air dried for 5 minutes. Yield: 19 mg (15%). 1H-NMR spectroscopy (in deuterated methanol) shows a molar ratio of Ezetimibe:proline of approximately 1:37.

Ezetimibe does not form a co-crystal with D-proline.

### Example 6

### Preparation at the 0.4 g Scale Starting with Crude Ezetimibe

460 mg of crude Ezetimibe (mixture of hydrate and anhydrate forms, containing about 10% of the stereoisomeric impurity 1-(4-Fluorophenyl)-3(R)-[3-(4-fluorophenyl)-3(R)-hydroxypropyl]-4(S)-(4-hydroxyphenyl)azetidin-2-one) and 116 mg of L-proline (Sigma #81709) are dissolved in 4.0 mL ethanol (≧99.8%) at 70° C., stirred at 70° C. for 30 minutes and cooled to 50° C. while stirring with a magnetic stirrer. The solution is seeded with approx. 5 mg of the 1:1 co-crystal of Ezetimibe with L-proline. 4.0 mL heptane are added to the turbid solution. The suspension formed is cooled to 40° C. and seeded again with approx. 5 mg of the 1:1 co-crystal of Ezetimibe with L proline. The suspension is further cooled to 27° C., sonicated for 1 minute and diluted with 8.0 mL of ethanol 99.8%):heptane 1:1 v/v. The suspension is seeded again with approx. 2 mg of the 1:1 co-crystal of Ezetimibe with L-proline and stirred for 14 hours at room temperature. The suspension is then filtered, air dried for 5 minutes, dried at room temperature/30 mbar for 30 minutes and at 50° C./30 mbar for 0.5 hour. Yield: about 300 mg (51%). 1 H-NMR spectroscopy (in deuterated methanol) indicates a molar ratio of Ezetimibe to L-proline of 1:1. Strong reduction of the amount of the impurity is confirmed by HPLC (method as described by Filip et al., J. Molec. Struct. 991, 162 (2011)).

### BRIEF DESCRIPTION OF FIGURES

FIG. 1: Powder X-ray diffraction pattern of the crystalline composition

FIG. 2: FT Raman spectrum of the crystalline composition in the range from 1800 to 200 cm⁻¹.

FIG. 3: FT Raman spectrum of the crystalline composition in the range from 3200 to 2700 cm⁻¹

FIG. 4: DVS of the crystalline composition of the present invention (bold line) and crystalline anhydrous Ezetimibe (Form A of WO 06/060808; dashed line), y-axis left. The measurement program (surrounding relative humidity) is given by the dot-dashed line and y-axis (right).

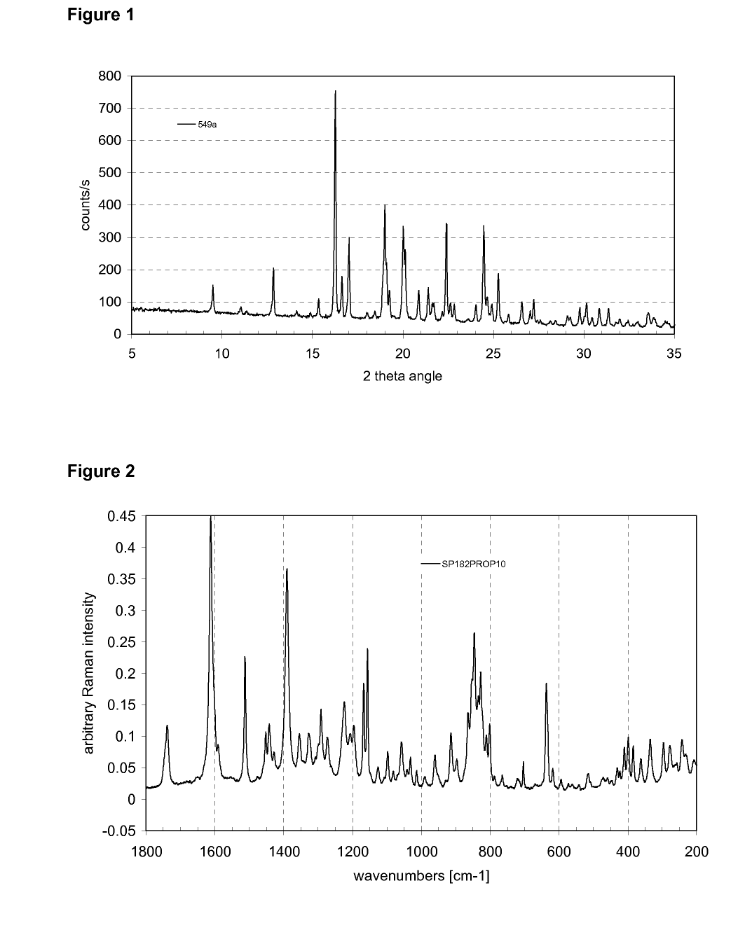

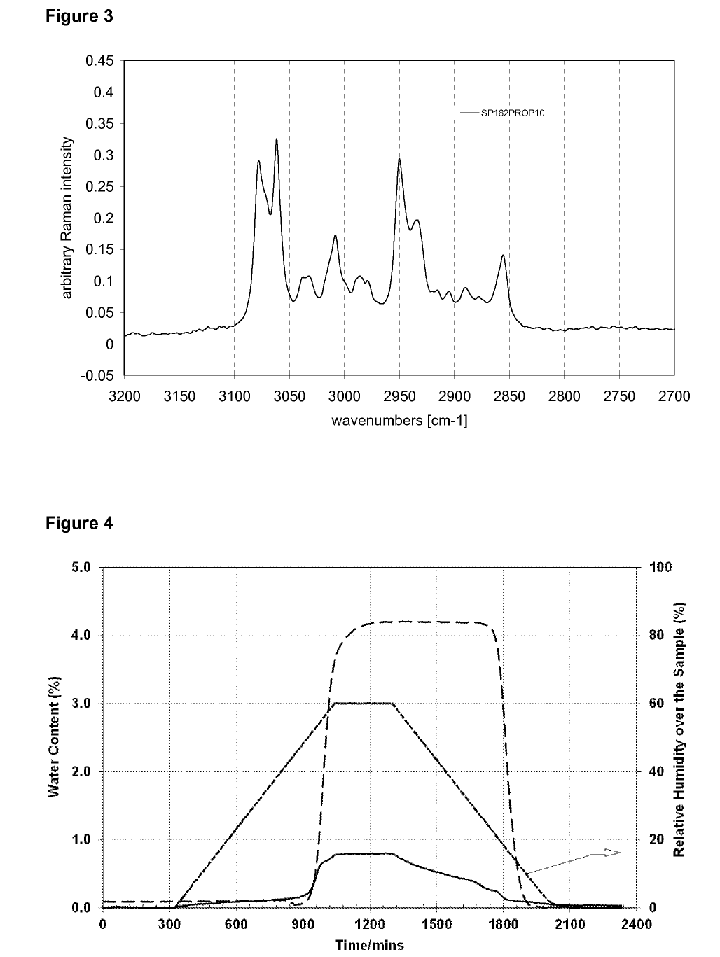

1. A composition, comprising:
a compound of formula 1: 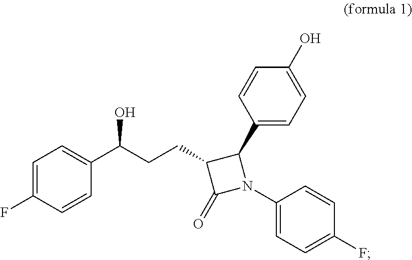 [SMILES: O=C1[C@H](CC[C@H](O)c2ccc(F)cc2)[C@@H](c2ccc(O)cc2)N1c1ccc(F)cc1]
and
proline, or a proline derivative.

2. The composition according to claim 1 which is a crystalline composition comprising a mixture of
the compound of formula 1: 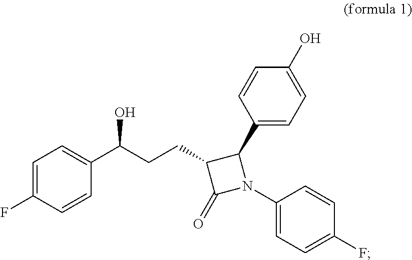 [SMILES: O=C1[C@H](CC[C@H](O)c2ccc(F)cc2)[C@@H](c2ccc(O)cc2)N1c1ccc(F)cc1]
and
the proline or the proline derivative.

3. The composition according claim 1 which forms a single crystalline phase (co-crystal).

4. The composition of claim 1, comprising the compound of formula 1 and proline, or a hydrate/solvate of said composition.

5. The composition of claim 1, wherein a molar ratio of the compound of formula 1 and the proline or the proline derivative is the range of from 1:0.5 to 1:2.1.

6. The composition of claim 1, having an XRPD pattern with characteristic peaks (expressed in 2θ±0.2°2θ; CuKα radiation) at 16.3, 17.0, 19.0, 20.0, 22.4, and 24.5°.

7. The composition according to claim 6, having an XRPD pattern with characteristic peaks (expressed in 2θ±0.2°2θ; CuKα radiation) at 9.5, 12.9, 16.3, 16.7, 17.0, 19.0, 20.0, 22.4, 24.5, and 25.3°.

8. The composition of claim 1, comprising the compound of formula 1 and (S)-proline, said composition being a co-crystal wherein a molar ratio of the compound of formula 1 and the (S)-proline is in the range of from 1:0.9 to 1:1.1.

9. A process for obtaining the composition of claim 2, the process comprising:
a) combining the compound of formula 1, or a mixture comprising the compound of the formula 1: 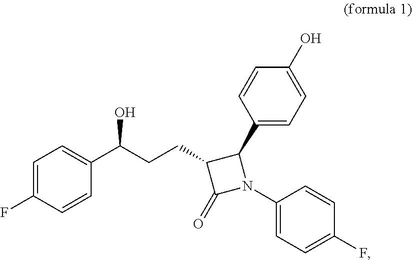 [SMILES: O=C1[C@H](CC[C@H](O)c2ccc(F)cc2)[C@@H](c2ccc(O)cc2)N1c1ccc(F)cc1]
with a solvent or a mixture of solvents, to form a mixture a);
b) adding the proline or the proline derivative to the mixture a) to obtain a composition b);
c) optionally concentrating the composition b) and/or adding an antisolvent;
d) crystallizing to form a suspension d);
e) optionally equilibrating the suspension d); and
f) isolating a precipitate formed from the step d) and/or the step e).

10. A process for purifying the compound of formula 1: 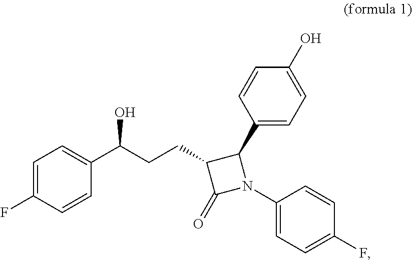 [SMILES: O=C1[C@H](CC[C@H](O)c2ccc(F)cc2)[C@@H](c2ccc(O)cc2)N1c1ccc(F)cc1]
the process comprising:
combining the composition of claim 1 with a solvent, to form a solution or dispersion; and
isolating the composition as a crystalline composition.

11. The process according to claim 9, wherein a molar ratio of the compound of formula 1 and the proline or the proline derivative is in the range from 1:0.5 to 1:3.

12. The process of claim 9, wherein proline is added in the step b).

13. The process of claim 9, wherein the solvent or the mixture of solvents is selected from the group consisting of a C1-C4 alcohol, a C3-C6 ketone, an ether, a C1-C4 alkylester, acetonitrile, a hydrocarbon, and a mixture thereof.

14. The process of claim 9, further comprising adding seed crystals.

15. A pharmaceutical composition, comprising the composition of claim 2 and optionally one or more pharmaceutically acceptable excipients.

16. The pharmaceutical composition according to claim 15, which is a solid having at least one characteristic peak in an x-ray powder diffractogram (expressed in 2θ±0.2°2θ (CuKα radiation)) selected from the group consisting of 12.9, 16.3, 17.0, 19.0, 20.0, 22.4, and 24.5°.

17. The composition of claim 1, comprising at least one proline derivative selected from the group consisting of 2-methylproline, 3-methylproline, 4-methylproline, 5-methylproline, N-methylproline, proline methylester, 4-hydroxyproline, and 3,4-dehydroproline.

18. The composition of claim 1, comprising at least one proline derivative selected from the group consisting of (S)-proline, 2-methyl-(S)-proline, 3-methyl-(S)-proline, 4-methyl-(S)-proline, 5-methyl-(S)-proline, N-methyl-(S)-proline, (S)-proline methylester, 4-hydroxy-(S)-proline, and 3,4-dehydro-(S)-proline.

19. The composition of claim 2, comprising a proline derivative selected from the group consisting of 2-methylproline, 3-methylproline, 4-methylproline, 5-methylproline, N-methylproline, proline methylester, 4-hydroxyproline, and 3,4-dehydroproline.
